(19) United States
(12) Patent Application Publication
(10) Pub. No.: US 2017/0209383 A1
(43) Pub. Date: Jul. 27, 2017

(54) COMPOSITION FOR NAP PROMOTION

(71) Applicant: ALVA-AMCO PHARMACAL COMPANIES, INC., Niles, IL (US)

(72) Inventor: Micah S. Gerchenson, Chicago, IL (US)

(21) Appl. No.: 15/481,532

(22) Filed: Apr. 7, 2017

Related U.S. Application Data
(63) Continuation of application No. PCT/US2015/054389, filed on Oct. 7, 2015.
(60) Provisional application No. 62/061,563, filed on Oct. 8, 2014.

(51) Int. Cl. A61K 9/48; A61K 36/35; A61K 36/82; A61K 36/67; A61K 31/522; A61K 31/4045; A61K 36/185

(52) U.S. Cl. CPC A61K 9/4825; A61K 31/4045; A61K 36/35; A61K 36/185; A61K 36/67; A61K 31/522; A61K 36/82

(57) ABSTRACT

The present invention relates to an oral formulation for promoting napping in a subject. The formulation may comprise an outer layer comprising a sedative and/or relaxant, and an inner layer comprising a stimulant.

### FIELD OF THE INVENTION

The present invention relates to compositions for promoting napping and methods for using the compositions.

### BACKGROUND OF THE INVENTION

Many Americans today are not getting the recommended amount of sleep each night. According to the American Sleep Association, every year approximately 40 million Americans, if not more, are afflicted by chronic, long-term sleep disorders. Spending related to sleep has increased 8.8% annually since 2008, reaching about $32 billion in 2012. In 2012, 73% of American Internet users went online to research health information, and 43% looked specifically for sleep remedies. Problems associated with sleep deficiencies extend well beyond fatigue and crankiness. Recent sleep studies have linked insufficient sleep to a host of problems including hypertension, depression, anxiety, diabetes, improper immune functioning, forgetfulness, clumsiness, jumpiness and even things like teen sports injuries. Forty-five percent of adults surveyed by the National Sleep Foundation reported that they “rarely” or “never” get a good night's sleep in any given week. This has resulted in consumers compensating either by ingesting sleep aids to help them fall asleep and stay asleep for eight hours through the night and/or consuming large amounts of caffeine to stay awake at work or at school.

The OTC sleep-aid market has continued to grow in recent years, hitting $759 MM in sales in 2013. The category is divided into drug and supplement ingredients: most commonly, diphenhydramine (an antihistamine) and melatonin, respectively. Diphenhydramine sleep aids are known to help produce long, drug-induced periods of sleep that typically last between 6-8 hours. Additionally, antihistamine-based sleep aids often have a paradoxical effect, especially for those older than 65, and can cause wakefulness. Melatonin in common doses found over-the-counter such as 3 mg-10 mg are also often consumed to help users get a full night of sleep. However, all existing sleep aids can cause drowsiness that can last between three and eight hours and cannot be taken after 4 AM for those needing to get up for work in the morning. For example, the directions for melatonin-based MidNite® state: “Do not drive or operate equipment for three hours after taking.”

Americans are also consuming caffeine in record numbers, and the energy category has exploded in recent years. Globally, the energy drinks and shots business market grew into a $27.5 billion business last year from $3.8 billion in 1999 and U.S. Sales of energy drinks and shots in 2013 totaled about $9B—up 17% versus 2012. Energy shots are expected to grow 11-12% annually until 2016—even though 59% of energy drink users are concerned about safety. 95% of adults drink at least one caffeinated beverage a day and average 3.1 beverages per workday. However, simply caffeinating a tired brain can actually decrease memory performance and can make one feel more wired, while also making one more prone to make mistakes.

Sleep-deprived Americans continue to compensate for poor sleep with naps. Forty-two percent of adults 18-64 report napping at least once a week, with an average of 1.4 naps taken during the workweek and one nap on non-work-days. Napping has plenty of proven benefits including, relaxation, Reduced fatigue, Increased alertness, Improved mood, Improved mental performance, Enhanced creativity, Improved reaction, Better memory, Less confusion, Fewer accidents and mistakes, The recommended length of nap is less than 45 minutes. A nap between 15 and 30 minutes is also known as a “power nap” and is recommended for short-term alertness and energy, improving alertness and performance, and helping the subject feel more alert and less sleepy even if they didn't sleep much the previous night. Short naps also do not interfere with nighttime sleep. However, many adults may find it difficult to fall asleep quickly enough during the day in order to achieve a “power nap” in their allotted nap schedule. Even among those whose schedules can accommodate a longer nap, many are reluctant because they fear the “sleep inertia” or grogginess that can set in after naps.

### SUMMARY OF THE INVENTION

Provided herein is an oral formulation comprising an outer layer, which may comprise a sedative and optionally a relaxant, and an inner layer comprising a stimulant. The sedative, relaxant, and stimulant may be from a natural source. The inner layer may comprise a capsule and the outer layer may comprise a coating. The sedative may comprise melatonin, valerian root extract, hops extract, or kava extract. The relaxant may comprise chamomile extract, lavender extract, lemon balm extract, or passion flower extract. The stimulant may comprise caffeine, which may be from a green tea extract, guarana extract, coffee extract, or yerba mate extract. The sedative may comprise melatonin, the relaxant may comprise chamomile extract, and the stimulant may comprise a green tea extract. The formulation may comprise 1 mg melatonin, 1 mg chamomile extract, and 500 mg green tea extract.

Also provided herein is a coated capsule comprising a liquid fill, a shell surrounding the liquid fill, and a coating around the shell. The liquid fill may comprise the stimulant, a liquid vehicle, a plasticizer, and water. The shell may comprise gelatin, an opacifier, optionally a colorant, and optionally water. The coating may comprise a cellulose derivative, a polyether, a mineral oil, the sedative, and the relaxant. The liquid vehicle may comprise a vegetable oil, and the plasticizer may comprise glycerol. The opacifier may comprise titanium dioxide. The cellulose derivative may comprise hypromellose and the polyether may comprise PEG 400. The liquid fill may comprise 500 mg green tea extract, 300 mg vegetable oil, 50 mg glycerol, and water. The shell may comprise 265 mg gelatin, 10 mg titanium dioxide, and 1 mg colorant. The coating may comprise 52 mg hypromellose, 28 mg PEG 400, 6 mg mineral oil, 1 mg melatonin, and 1 mg chamomile extract.

Further provided herein is a method of promoting napping in a subject, which comprises administering the oral formulation or the coated capsule to a subject in need thereof.

### DETAILED DESCRIPTION

The inventors have discovered that an oral formulation comprising an outer portion comprising a sedative and/or relaxant, and an inner portion comprising a stimulant, is surprisingly effective in promoting napping in a subject for about 30-45 minutes, with no nap hangover or grogginess. When the formulation is swallowed by the subject, the outer portion may dissolve in the mouth or stomach, where the released sedative and/or relaxant may aid in relaxation and restful sleep. About 30 minutes after the formulation has been swallowed, the inner portion may dissolve, releasing the stimulant into the stomach. The stimulant may be absorbed from the stomach and have a pharmacological effect within about 45-60 minutes after the formulation is consumed. The formulation may enhance the nap experience of the subject by improving the quality of sleep, while delivering an energy boost as the subject awakens.

The outer portion helps the subject relax to begin their nap and to help improve the quality of their nap; the inner core then releases a time-delayed kick of a natural stimulant to help clear the mind 30-45 minutes later when the subject is awoken. The subject then awakes refreshed and not groggy while still enjoying the proven benefits of a short nap. PowerNap is non-addicting and is designed for improving short-duration naps that are more restful for the brain than purely consuming caffeine, while avoiding the 3-8 hours of grogginess that can ensue from traditional sleep aids.

### 1. Definitions.

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

### 2. Nap-Promoting Formulation

Provided herein is a formulation for promoting napping in a subject in need thereof. The napping may not induce grogginess. The formulation may be an oral formulation, and may comprise an inner portion and an outer portion. The inner portion may comprise a stimulant and the outer portion may comprise a sedative and/or a relaxant. The outer portion may begin dissolving in the subject's mouth and release the sedative and/or relaxant, thereby assisting the subject in achieving restful sleep within about 15 minutes of consuming the formulation. The inner portion may dissolve in the subject's stomach within about 30 minutes, which may release the stimulant. The stimulant may have a pharmacological effect within 15 minutes of being absorbed in the subject's stomach, thereby causing the subject to feel energized. The formulation may thus promote a nap of about 30-45 minutes in the subject.

a. Stimulant

The stimulant may comprise caffeine, which may comprise a caffeine from a natural source. The source of the caffeine may be a green tea extract, guarana extract, coffee extract, or yerba mate extract, or a combination thereof. While the stimulant may comprise caffeine in particular, the stimulant may also comprise, or comprise an extract of, Acetyl-L-Carnitine/L-Carnitine, Arginine, Biotin, Betel nut, B-vitamins, Cacao, Cayenne, Chia seeds, Chlorella, Chlorophyll, Chromium picolinate, Coconut oil, Coenzyme Q-10, Cordyceps, Damiana, Dehydroepiandrosterone (DHEA), Eluthero, Ephedra, Fo-Ti, Free form amino acids, Ginkgo, Ginseng, which may be Asian or Red, Gotu kola/kula, Grape seed, Green coffee, Guayusa, Holy basil extract, Hoodia, Indian ginseng, Iodine, L-Theanine, L-Tyrosine, Maca, Mucuna pruriens, Niacin, Rhodiola rosea, Sage, Selenium, St. John's Wort, Taurine, Tea (Cammillia sinensis), Theobroma cacao, Tobacco, Xanthine, or Yohimbe.

b. Relaxant

The relaxant may comprise a natural relaxant, which may comprise a chamomile extract, lavender extract, lemon balm extract, or passion flower extract, or a combination thereof. While the relaxant may comprise a chamomile extract in particular, the relaxant may also comprise, or comprise an extract of, Ashwagandha root, Benzoin Resin, Bergamot Oil, Boswellia (Frankincense), Calamus root, Calcium, California poppy, Camphor, Catnip, Cedarwood, Celandine, Chaste tree/vitex/chasteberry, Cherries/cherry juice, Clary Sage, Coriander, Cornflowers, Corydalis, DHEA, Diphenhydramine, Dong quai, Fumitory, Geranium, Hemp Oil, Hyssop, Inositol, Jamaican Dogwood, Jasmine Flower Oil, Juniper Berries, Linden, Lotus, Magnesium, Marjoram, Melatonin, Milk peptides, Motherwort, Myrrh, Nard Oil, Niacinamide, Orange, Patchouli, Periwinkle, Potassium, Red clover, Rose, Sandalwood, Schisandra, Skullcap, Stevia herb, Sweet Woodruff, Tryptophan, Wild Cherry Bark, Wild lettuce, Wintergreen, or Ylang-Ylang.

c. Sedative

The sedative may comprise melatonin, which may comprise a melatonin from a natural source. The sedative may also be a valerian root extract, a hops extract, or a kava extract, or a combination thereof.

d. Inner Portion

The inner portion may comprise a capsule, and the outer portion may comprise a capsule coating. The capsule may be a softgel or a hard-shell capsule. The capsule may comprise a liquid fill, a powder fill, or a semi-solid fill; and a shell.
(1) Liquid Fill

The liquid fill may comprise the stimulant, a liquid vehicle, a plasticizer, a surfactant, water, a solubilizing agent, and a suspending agent.

(a) Liquid Vehicle

The liquid vehicle may comprise a lipophilic liquid or a semi-solid. The lipophilic liquid may comprise a vegetable oil and/or polyether. The semi-solid may comprise a hydrogenated oil, such as castor oil, and/or a wax such as bees wax.

(b) Plasticizer

The plasticizer may comprise glycerol, glycerin, sorbitol, or propylene glycol, or a combination thereof.

(c) Surfactant

The surfactant may comprise a lecithin, sorbitol, polysorbate, or sorbitan, or a combination thereof.

(d) Solubilizing Agent

The solubilizing agent may comprise a beeswax or a mono-, di-, or triglyceride, or a combination thereof.

(e) Suspending Agent

The suspending agent may comprise maltodextrin, sodium alginate, or xanthan gum, or a combination thereof.

(2) Powder Fill

The powder fill may comprise the stimulant, a diluent, an anti-caking agent, and a lubricant.

(a) Diluent

The diluent may comprise dicalcium phosphate, lactose, maltodextrin, microcrystalline cellulose, or a starch, or a combination thereof.

(b) Anti-Caking Agent

The anti-caking agent may comprise magnesium silicate, silica gel, or talc, or a combination thereof.

(c) Lubricant

The lubricant may comprise hydrogenated vegetable oil, magnesium stearate, mineral oil, or stearic acid, or a combination thereof.

(3) Semi-Solid Fill

The semi-solid fill may comprise the stimulant, a semi-solid vehicle, a surfactant, and an emulsifying agent.

(a) Semi-Solid Vehicle

The semi-solid vehicle may comprise hydrogenated palm oil, hydrogenated castor oil, cetyl alcohol, cetosteryl alcohol, a stearoyl polyoxylglyceride, a laurolyl polyoxyglyceride, or a combination thereof.

(b) Surfactant

The surfactant may be a lecithin, sorbitol, polysorbate, or sorbitan, or a combination thereof.

(c) Emulsifying Agent

The emulsifying agent may be polyethylene glycol, or poloxamer, or a combination thereof.

(4) Shell

The shell may comprise a gelling agent, the plasticizer, an opacifier, a colorant, and water.

(a) Gelling Agent

The gelling agent may comprise gelatin, a plant polysaccharide, a carrageenan, a modified starch, a cellulose or derivative thereof, or a combination of the foregoing. The modified starch may comprise starch hydrolysate. The cellulose derivative may comprise hypromellose or methylcellulose, or a combination thereof.

(b) Opacifier

The opacifier may comprise titanium dioxide.

(c) Plasticizer

The plasticizer may comprise glycerol, glycerin, sorbitol, or propylene glycol, or a combination thereof.

e. Outer Portion

The outer portion may comprise a coating, which may comprise the sedative and/or relaxant.

(1) Coating

The coating may comprise the sedative and/or the relaxant, a cellulose or derivative thereof, a polyether, a mineral oil, water, a plant resin or protein, and a surfactant. The polyether may comprise a polyethylene glycol, which may comprise a low molecular weight polyethylene glycol such as a PEG 300-600 or PEG 400, or a high molecular weight polyethylene glycol such as a PEG 4000-10,000, or a combination thereof.

f. Doses

The formulation may comprise 50-1000 mg of the stimulant, and more specifically may comprise 200-800, 300-600, 50, 100, 150, 200, 250, 300, 350, 400, 450, 500, 550, 600, 650, 700, 750, 800, 850, 900, 950, or 1000 mg of the stimulant. The formulation may also comprise 0.1-10 mg of the sedative, and more specifically may comprise 0.5-1.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg of the sedative. The formulation may also comprise 0.1-10 mg of the relaxant, and more specifically may comprise 0.5-1.0, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mg of the relaxant.

If the formulation is a capsule, the capsule may comprise the ingredients listed in Table 1.

TABLE 1

| Ingredient | Amount (mg) |
|---|---|
| Green tea extract | 100-1000 |
| Vegetable oil | 100-1000 |
| Gelling agent | 10-1000 |
| Plasticizer | 1-500 |
| Opacifier | 0.1-50 |
| Water | 0.1-50 |
| Colorant | 0-50 |

and the coating may comprise the ingredients listed in Table 2.

TABLE 2

| Ingredient | Amount (mg) |
|---|---|
| Cellulose derivative | 10-200 |
| Polyethylene glycol | 1-100 |
| Mineral oil | 0.1-10 |
| Sedative | 0.1-50 |
| Relaxant | 0.1-50 |

3. Method of nap promotion

Provided herein is a method of promoting a nap in a subject. The nap may not induce grogginess. The method may comprise administering a formulation described herein to a subject in need thereof.

The present invention has multiple aspects, illustrated by the following non-limiting examples.

### EXAMPLE 1

### Oral Capsule for Promoting Naps

This example describes a formulation of a coated capsule for promoting naps. The capsule contains the ingredients listed in Table 3.

TABLE 3
Capsule ingredients

| Ingredient | Amount (mg) |
|---|---|
| Green tea extract | 500 |
| Vegetable oil | 300 |
| Gelatin | 265 |
| Glycerol | 50 |
| Titanium dioxide | 10 |
| Purified water | 6 |
| Coloring | 1 |

The coating contains the ingredients listed in Table 4.

TABLE 4
Coating ingredients

| Ingredient | Amount (mg) |
|---|---|
| Hypromellose | 52 |
| Polyethylene glycol 400 | 28 |
| Mineral oil | 6 |
| Melatonin | 1 |
| Chamomile extract | 1 |

The formulation is made by mixing green tea extract, vegetable oil, glycerol, and purified water, with heating as necessary, to form a liquid fill solution or uniform suspension. The gelatin is heated to obtain the proper viscosity, and then the titanium oxide and coloring agents are added to form the capsule shell. Additional water is added as needed to obtain the desired viscosity for the shell. The gelatin is then formed into ribbons on encapsulation equipment, and the liquid is encapsulated within the gelatin using Oval No. 12 dies. The resulting softgel capsules are dried at the appropriate temperature and airflow.

The coating ingredients from Table 4 are mixed with water and heated as necessary to form a solution. The solution is applied to the softgel capsules using a 60″ coating pan operated at the appropriate airflow, input temperature, and exhaust temperature.

### EXAMPLE 2

### Core Tablet

A core tablet comprising guarana extract, lactose, microcrystalline cellulose, sodium starch glycolate, hypromellose, and magnesium stearate, as shown in Table 5, was tested for its caffeine dissolution profile. The results are shown in Table 6.

TABLE 5
Tablet ingredients

| Ingredient | Amount (mg) |
|---|---|
| Guarana extract | 250 |
| Lactose | 245 |
| Microcrystalline cellulose | 476 |
| Sodium starch glycolate | 15 |
| Hypromellose | 10 |
| Magnesium stearate | 4 |

TABLE 6
Tablet dissolution

| Time (hr) | % dissolution |
|---|---|
| 1 | 34.1 |
| 2 | 50.3 |
| 3 | 67.0 |

Because less than 90% of the caffeine was dissolved after 2 hours, the core tablet formulation is not well-suited for use in promoting non-grogginess-inducing naps.

### EXAMPLE 3

### Core Tablet

A core tablet comprising guarana extract, calcium sulfate, croscarmellose sodium, magnesium stearate, microcrystalline cellulose, polyvinyl pyrrolidone, and sodium starch glycolate, as shown in Table 7, was tested for its caffeine dissolution profile. The results are shown in Table 8.

TABLE 7
Tablet ingredients

| Ingredient | Amount (mg) |
|---|---|
| Guarana extract | 250 |
| Calcium sulfate | 200 |
| Croscarmellose sodium | 25 |
| Magnesium stearate | 1 |
| Microcrystalline cellulose | 504 |
| Polyvinyl pyrrolidone | 10 |
| Sodium starch glycolate | 10 |

TABLE 8
Tablet dissolution

| Time (hr) | % dissolution |
|---|---|
| 0.5 | 31.0 |
| 1 | 57.5 |
| 2 | 87.5 |

Because less than 90% of the caffeine was dissolved after 1 hour, the core tablet formulation is not well-suited in promoting non-grogginess-inducing naps.

1. An oral formulation comprising an outer layer comprising a sedative and optionally a relaxant, and an inner layer comprising a stimulant.

2. The formulation of claim 1, wherein the inner layer comprises a capsule and the outer layer comprises a coating.

3. The formulation of claim 2, wherein the sedative is selected from the group consisting of melatonin, valerian root extract, hops extract, and kava extract.

4. The formulation of claim 2, wherein the relaxant is selected from the group consisting of a chamomile extract, lavender extract, lemon balm extract, and passion flower extract.

5. The formulation of claim 2, wherein the stimulant comprises caffeine.

6. The formulation of claim 5, wherein the source of the caffeine is selected from the group consisting of a green tea extract, guarana extract, coffee extract, and yerba mate extract.

7. The formulation of claim 2 comprising a relaxant, wherein the sedative comprises melatonin, the relaxant comprises a chamomile extract, and the stimulant comprises caffeine from a green tea extract.

8. The formulation of claim 7, wherein the formulation comprises 1 mg melatonin, 1 mg chamomile extract, and 500 mg green tea extract.

9. A coated capsule comprising a liquid fill, a shell surrounding the liquid fill, and a coating around the shell, wherein the liquid fill comprises a stimulant, a liquid vehicle, a plasticizer, and water; the shell comprises a gelatin, an opacifier, optionally a colorant, and optionally water; and the coating comprises a cellulose derivative, a polyether, a mineral oil, a sedative, and a relaxant.

10. The capsule of claim 9, wherein the stimulant comprises caffeine.

11. The capsule of claim 10, wherein the caffeine is from a green tea extract.

12. The capsule of claim 9, wherein the sedative comprises melatonin.

13. The capsule of claim 9, wherein the relaxant comprises a chamomile extract.

14. The capsule of claim 9, wherein the liquid vehicle comprises a vegetable oil and the plasticizer comprises glycerol.

15. The capsule of claim 9, wherein the opacifier comprises titanium dioxide.

16. The capsule of claim 9, wherein the cellulose derivative comprises hypromellose and the polyether comprises PEG 400.

17. A coated capsule comprising a liquid fill, a shell surrounding the liquid fill, and a coating around the shell, wherein the liquid fill comprises 500 mg green tea extract, 300 mg vegetable oil, 50 mg glycerol, and water; the shell comprises 265 mg gelatin, 10 mg titanium dioxide, and 1 mg colorant; and the coating comprises 52 mg hypromellose, 28 mg PEG 400, 6 mg mineral oil, 1 mg melatonin, and 1 mg chamomile extract.

18. A method of promoting napping in a subject, comprising administering the oral formulation of claim 1 to a subject in need thereof.

19. A method of promoting napping in a subject, comprising administering the coated capsule of claim 9 to a subject in need thereof.

20. A method of promoting napping in a subject, comprising administering the coated capsule of claim 17 to a subject in need thereof.
